(19) United States
(12) Patent Application Publication
(10) Pub. No.: US 2021/0315250 A1
(43) Pub. Date: Oct. 14, 2021

(54) COMBINATION BROAD-SPECTRUM ANTIVIRAL NATURAL EXTRACT SUPPLEMENTS

(71) Applicant: Mark Edward Fenzl, Sarasota, FL (US)

(72) Inventor: Mark Edward Fenzl, Sarasota, FL (US)

(21) Appl. No.: 17/175,613

(22) Filed: Feb. 13, 2021

Related U.S. Application Data
(60) Provisional application No. 63/100,924, filed on Apr. 8, 2020.

(51) Int. Cl. A23L 33/125; A61K 45/06; A23L 33/115

(52) U.S. Cl. CPC A23L 33/125; A23L 33/115; A61K 45/06

(57) ABSTRACT

The present technology may include combining different non-synthetic products to form a broad-spectrum antiviral supplement that inhibit or kill viruses. The antiviral supplement can be added to a shake, juice, or food to include antiviral properties. In one embodiment, the supplement may be configured to be admixed in the product. The product may include at least four ingredients selected from a first group, a second group, and a third group. In particular, the supplement may include at least four ingredients with two ingredients selected from the first group. Sweeteners including honey, artificial honey, molasses, or syrup combined with natural preservatives can also be used to provide broad-spectrum antiviral protection.

### CROSS-REFERENCE TO RELATED APPLICATIONS

This application claims the benefit of U.S. Provisional Application No. 63/100,924, filed on Apr. 8, 2020. The entire disclosure of the above application is incorporated herein by reference.

### FIELD

The present disclosure relates to antivirals and, more particularly, to antiviral supplements.

### BACKGROUND

This section provides background information related to the present disclosure which is not necessarily prior art.

Antivirals kill and inhibit viruses that can lead to infection or death. Historically, teas have been protective from viral infections. Prior to development of the scientific method, there was no means to determine the effectiveness of teas to protect from viral infection, and prior to the invention of the electron microscope, viruses could not even be observed. Cultivars of domesticated herbs, flowers, fruit, and berries were cultivated for their size and strength of their smell and taste. Chemical concentration was determined by taste and smell and often herbs with stronger taste were more likely to preserve food for a longer period of time and flowers and fruit with a stronger scent were more likely to have stronger antimicrobial properties. People that consumed natural antivirals were more likely to live through viral epidemics than those that did not. This knowledge was passed down to the next generation.

Currently, antivirals can be tested for efficacy. There are commercial antiviral products that use concentrated chemicals for the treatment of infections, but there are a wide variety of viruses that are constantly mutating and can evade antiviral medications. This is also an issue with immunizations. Immunizations can be effective in preventing viral infections, but certain viruses have not been eradicated due to people refusing vaccinations as well as populations that do not have access to vaccines.

One difficulty with using plant products as antivirals is that plant products that are resistant to some microorganisms are susceptible to spoilage by other microorganisms. One way to avoid this problem is limiting the amount of water available in the product by using concentrated plant products and mixing the plant products with materials that lower water activity level. Water activity level is the amount of free water in a product. Examples of materials that lower water activity level include glycerol. These materials bind to water to prevent the availability of free water that bacteria need to grow and multiply. Another way to protect the product is to change the pH or acidity to a high level or a very low level. One example is that lowering the pH of a product inhibits bacteria like Pseudomonas. One material that can lower the pH is citric acid.

Synthetic antivirals have been effective in the past at inhibiting and killing viruses at lower concentrations than plant products. After the development of synthetic antivirals, there was a loss in interest in using plant products as antibiotics. Over time, new viruses have been exposed to the public and other viruses have mutated with resistance to synthetic antivirals, rendering them ineffective.

With the advent of rapid mass public transportation and housing of the largest concentrations of people in major cities ever seen, the threat of pandemics has increased significantly. Pandemics have created a huge expense in health care, loss in wealth and income, and pain and suffering for those with infections that are resistant to treatment.

There is a continuing need for a broad-spectrum antiviral supplement. Desirably, the supplement is manufactured by combining concentrated, non-synthetic plants products.

### SUMMARY

In concordance with the instant disclosure, a broad-spectrum antiviral supplement that is manufactured by combining concentrated, non-synthetic plants products, has surprisingly been discovered.

The present technology may include combining different non-synthetic products to form a broad-spectrum antiviral supplement that inhibit or kill viruses. The antiviral supplement can be added to a shake, juice, or food to include antiviral properties.

In one embodiment, the supplement may be configured to be admixed in the product. The product may include at least five ingredients selected from a first group, a second group, and a third group. In particular, the supplement may include at least two ingredients selected from the first group. The first group can contain anethole, benzaldehyde, carvacrol, caryophyllene, cineole, cinnamaldehyde, citral, citronellal, curcumin, farnesol, eugenol, limonene, linalool, methylglyoxal, pinene, terpineols, thujone, thymol, and vanillin at a concentration between 50 and 50,000 parts per million in the product. The second group can contain betulinic acid, caprylic acid, chebulic acid, ferulic acid, flavonols, gingerol, hesperidin, isothiocyanates, mangostin, luteolin, myristic acid, oleuropein, pectolinarin, piperine, protocatechuic acid, pterostilbene, punicalagin, rhoifolin, sanguiin, and withanolides at a concentration between 100 and 100,000 parts per million in the product. The third group can contain acetic acid, citric acid, dextrose, fructose, glycerol, glycine, glucose, lactic acid, lactose, malic acid, salt, sorbitol, sucrose, and tartaric acid at a concentration sufficient to change a water activity level or pH of the product.

In a second embodiment, the supplement may be configured to be admixed in the product. The product may include at least four ingredients selected from a first group, a second group, and a third group. In particular, the supplement may include at least three ingredients selected from the first group, and at least one ingredient selected from each of the second group and the third group. The first group can contain anethole, benzaldehyde, carvacrol, caryophyllene, cineole, cinnamaldehyde, citral, citronellal, curcumin, eugenol, farnesol, geraniol, limonene, linalool, methylglyoxal, pinene, terpineols, thujone, thymol, and vanillin wherein each of the ingredients of the first group are present at a concentration of between 50 and 50,000 parts per million in the product. The second group can contain betulinic acid, caprylic acid, chebulic acid, ferulic acid, flavonols, gingerol, hesperidin, isothiocyanates, mangostin, luteolin, myristic acid, oleuropein, pectolinarin, piperine, protocatechuic acid, pterostilbene, punicalagin, rhoifolin, sanguiin, and withanolides at a concentration between 100 and 100,000 parts per million in the product. The third group can contain acetic acid, citric acid, dextrose, fructose, glycerol, glycine, glucose, lactic acid, lactose, malic acid, salt, sorbitol, sucrose, and tartaric acid at a concentration sufficient to change a water activity level or pH of the product.

In a third embodiment, the supplement may be configured to be admixed in the product. The product may include at least four ingredients selected from a first group, a second group, and a third group. In particular, the supplement may include at least three ingredients selected from the first group, and at least one ingredient selected from the third group. The first group can contain anethole, benzaldehyde, carvacrol, caryophyllene, cineole, cinnamaldehyde, citral, citronellal, curcumin, eugenol, farnesol, geraniol, limonene, linalool, methylglyoxal, pinene, terpineols, thujone, thymol, vanillin, and zingiberene wherein each of the ingredients of the first group are present at a concentration of between 50 and 50,000 parts per million in the product. The second group can contain betulinic acid, caprylic acid, chebulic acid, ferulic acid, flavonols, gingerol, hesperidin, isothiocyanates, mangostin, luteolin, myristic acid, oleuropein, pectolinarin, piperine, protocatechuic acid, pterostilbene, punicalagin, rhoifolin, sanguiin, and withanolides at a concentration between 100 and 100,000 parts per million in the product. The third group can contain acetic acid, citric acid, dextrose, fructose, glycerol, glycine, glucose, lactic acid, lactose, malic acid, propylene glycol, salt, sorbitol, sucrose, and tartaric acid at a concentration sufficient to change a water activity level or pH of the product.

The third embodiment uses honey as a natural sweetener. Honey, some molasses, and some syrups have natural antiviral properties along with low water activity that protects products from microbial contamination. Honey and honey substitutes can be used for antiviral and preservative effect.

Another ingredient, monolaurin, is added to the third embodiment. Monolaurin is a natural monoglyceride that inhibits or kills a broad-spectrum of microbes including bacterial spores, bacteria, fungi, and enveloped viruses. Coronavirus, for example, is an enveloped virus. Monoalaurin is more effective in an acidic environment and is synergistic in combination with the natural preservative sorbic acid. Monolaurin can also be used for antiviral and preservative effect.

Further areas of applicability will become apparent from the description provided herein. The description and specific examples in this summary are intended for purposes of illustration only and are not intended to limit the scope of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

The present disclosure relates to a supplement that is configured to be mixed into a product. Advantageously, the supplement may be a broad-spectrum antiviral supplement, that may inhibit viruses. The supplement may be manufactured by combining multiple, concentrated, non-synthetic products, as described in greater detail hereinbelow.

It is believed that the supplement of the present disclosure may be beneficial in inhibiting viruses due to a binding of the supplement to a docking site of the virus. It has been found that 3-dimensional imaging computer simulations may be developed to determine if the supplement may bind to the docking site of the virus. Further, the attraction, or binding energy, of the supplement to the docking site may be calculated. In one particular example, COVID-19 has a known docking site on a 3CLpro enzyme, which also called Main protease (M^{pro}). Therefore, in order to inhibit the COVID-19 virus, the supplement may desirably bind to the 3CLpro enzyme.

In one unlimiting embodiment, the supplement may be configured to be admixed in the product. The product may include at least five ingredients selected from a first group, a second group, a third group. In particular, the supplement may include at least two ingredients selected from the first group, and at least one ingredient selected from each of the second group, and the third group. A skilled artisan may select a suitable number of ingredients from each group, as desired.

At least two ingredients selected from the first group may each be present in the product at a concentration between 50 and 50,000 parts per million. The ingredients in the first group have been found to have antiviral properties at this concentration. The first group may contain anethole, benzaldehyde, carvacrol, caryophyllene, cineole, cinnamaldehyde, citral, citronellal, curcumin, farnesol, eugenol, limonene, linalool, methylglyoxal, pinene, terpineols, thujone, thymol, and vanillin. Anethole, eugenol, farnesol, and caryophyllene have all been shown to have significant antiviral properties. It has been found that chemicals naturally found in essential oils, such as, anethole, carvacrol, cineole, citronellal, and thymol may inhibit viruses at relatively low concentrations. Trans-anethole and caryophyllene have been shown to inhibit HSV-1. Curcumin has been shown to interact with both an ACE2 receptor and a viral S protein docking sites. Methylglyoxal has been shown to inhibit viruses, in particular, influenza.

The ingredients selected from the second group may be present in the product at a concentration between 100 and 100,000 parts per million. The ingredients in the second group have been found to have antiviral properties at this concentration. The second group may contain betulinic acid, caprylic acid, chebulic acid, ferulic acid, flavonols, gingerol, hesperidin, isothiocyanates, mangostin, luteolin, myristic acid, oleuropein, pectolinarin, piperine, protocatechuic acid, pterostilbene, punicalagin, rhoifolin, sanguiin, and withanolides. It has been shown that luteolin, savinin, and betulinic acid may inhibit the docking site for SARS-CoV 3CL. Flavonols, such as the catechin ECGC found in green tea, have been shown inhibit viruses from functioning at very low concentration for a wide variety of viruses. The natural fatty acid myristic acid has been shown to inhibit viruses, such as HIV and SVS. Myricetin has been shown to inhibit the docking site for SARS-CoV-2. Sanguiin, kaempferol, punicalagin, and protocatechuic acid have been shown to dock with a strong affinity to the COVID-19 main protease using computer modeling. The flavonoids herbacetin, rhoifolin, and pectolinarin have been shown to bind to SARS-CoV 3CL.

The binding energy in kcal/mol units of certain ingredients from the second group at the M^{PRO} docking site are shown in TABLE 1 hereinbelow.

TABLE 1

| | | | M^{PRO} |
|---|---|---|---|
| | | | Binding |
| Compound | Class | Subclass | Energy |
| Sanguiin | Sanguiin acid | Hydrobenzoic acids | −10.3 |
| Theaflavin | Flavonoids | Flavanols | −10.0 |
| Kaempferol | Flavonoids | Flavanols | −9.8 |
| Protocatechuic Acid | Phenolic acids | Hydroxybenzoic acids | −9.8 |
| Punicalagin | Phenolic acids | Hydroxybenzoic acids | −9.8 |
| Savinin | Lignin | Dibenzyl butyrolactone | −9.1 |
| Betulinic acid | Steroids | Pentacyclic triterpenoid | −8.2 |
| Luteolin | Flavanoid | Flavones | −11.45 |
| Apigenin | Flavanoid | Flavones | −10.82 |
| Oleuropein | Phenol | Iridoid | −10.59 |
| Quercetin | Flavanoid | Flavonol | −10.26 |
| Curcumin | Phenol | Curcuminoid | −10.03 |

The at least one ingredient selected from the third group may be present in the product at a concentration sufficient to change a water activity level of the product or a pH of the product. The third group may contain acetic acid, citric acid, dextrose, fructose, glycerol, glycine, glucose, lactic acid, lactose, malic acid, propylene glycol, salt, sorbitol, sucrose, and tartaric acid.

In a second embodiment, the supplement may be configured to be admixed in the product. The product may include at least five ingredients selected from a first group, a second group, and a third group. In particular, the supplement may include at least three ingredients selected from the first group, and at least two ingredients selected from the third group. A skilled artisan may select a suitable number of ingredients from each group, as desired.

The at least three ingredients selected from the first group may each be present in the product at a concentration between 50 and 50,000 parts per million. The ingredients in the first group have been found to have antiviral properties at this concentration. The first group may contain anethole, benzaldehyde, carvacrol, caryophyllene, cineole, cinnamaldehyde, citral, citronellal, curcumin, eugenol, farnesol, limonene, linalool, methylglyoxal, pinene, terpineols, thujone, thymol, and vanillin.

The at least two ingredients selected from the third group may be present in the product at a concentration sufficient to change a water activity level of the product or a pH of the product. The third group may contain acetic acid, citric acid, dextrose, fructose, glycerol, glycine, glucose, lactic acid, lactose, malic acid, propylene glycol, salt, sorbitol, sucrose, and tartaric acid.

In a third embodiment, the supplement may be configured to be admixed in the product. The product may include at least five ingredients selected from a first group, a second group, and a third group. In particular, the supplement may include at least two ingredients selected from the first group, and at least one ingredient selected from the second group, and at least two ingredients selected from the third group. Honey and monolaurin have been added to the third embodiment for antiviral and preservative effect. A skilled artisan may select a suitable number of ingredients from each group, as desired.

The at least two ingredients selected from the first group may each be present in the product at a concentration between 50 and 50,000 parts per million. The ingredients in the first group have been found to have antiviral properties at this concentration. The first group may contain anethole, benzaldehyde, carvacrol, caryophyllene, cineole, cinnamaldehyde, citral, citronellal, curcumin, eugenol, farnesol, geraniol, limonene, linalool, methylglyoxal, pinene, terpineols, thujone, thymol, and vanillin.

The at least one ingredient selected from the second group may be present in the product at a concentration between 100 and 100,000 parts per million. The ingredients in the second group have been found to have antiviral properties at this concentration. The second group may contain betulinic acid, caprylic acid, chebulic acid, ferulic acid, flavonols, gingerol, hesperidin, isothiocyanates, luteolin, mangostin, myristic acid, oleuropein, pectolinarin, piperine, protocatechuic acid, pterostilbene, punicalagin, rhoifolin, sanguiin, and withanolides.

The at least two ingredients selected from the third group may be present in the product at a concentration sufficient to change a water activity level of the product or a pH of the product. The third group may contain acetic acid, citric acid, dextrose, fructose, glycerol, glycine, glucose, lactic acid, lactose, malic acid, propylene glycol, salt, sorbitol, sucrose, and tartaric acid at a concentration sufficient to change a water activity level or pH of the product.

In the third embodiment honey is used as a natural antiviral sweetener. But another natural antimicrobial sweetener, such a pomegranate molasses, may be substituted. A skilled artisan may make a substitution as desired.

Also in the third embodiment, monolaurin, a natural monoglyceride is added for additional antiviral effect. Another monoglyceride may be substituted by a skilled artisan.

The product, in which the supplement may be admixed, may be selected from the group consisting of a shake, thickened drink, weight loss drink, weight gain drink, milk, chocolate milk, juice, tea, carbonated water, water, honey, molasses, syrup, sauce, dressing, pudding, confection, or food, as non-limiting examples. Advantageously, the supplement may be admixed into commonly consumed household food items to provide the antiviral benefits of the supplement to a user.

In one non-limiting example, the product may be a chocolate syrup, which may be mixed with milk to create an antiviral chocolate milk. The chocolate syrup may include cocoa at a product concentration of 10,000 parts per million (1%), cinnamaldehyde at a product concentration of 300 parts per million (0.03%), eugenol at a concentration of 400 parts per million (0.04%), gum arabic at a concentration of 2,500 parts per million (0.25%), vanillin at a concentration of 250 parts per million (0.025%), myristic acid at a concentration of 2500 parts per million (0.25%), and corn syrup at a concentration of 150,000 parts per million (15%). It should be appreciated that cocoa, vanillin, and cinnamaldehyde have a synergistic antimicrobial effect.

In a second non-limiting example, the product is a coffee creamer, which may be mixed with coffee to create an antiviral coffee beverage. The coffee creamer may include the supplement with dehydrated milk at a product concentration of 20,000 parts per million (2%), bitter almond oil at a concentration of 1,000 parts per million (0.1%), star anise essential oil at a product concentration of 500 parts per million (0.05%), vanillin at a product concentration of 250 parts per million (0.025%), and lactic acid at a product concentration of 3000 parts per million (0.3)%.

In a third non-limiting example, the product may be a morning meal replacement shake. The shake may include pomegranate extract at a product concentration of 20,000 parts per million (2%), citric acid at a product concentration of 3,000 parts per million (0.3%), citral at a product concentration of 400 parts per million (0.04%), monolaurin at a product concentration of 10,000 parts per million (1%), methylglyoxal at a product concentration of 350 parts per million (0.035%), and honey at a product concentration of 20,000 parts per million (2%).

It may be beneficial to militate against microbial growth in the supplement. Microbial growth may be controlled by limiting the amount of water in the product by using concentrated plant products or admixing the plant products with plant materials that lower water activity level. Water activity level is the amount of free water in a product. Examples of materials that lower water activity level include glycerol or citric acid. These materials have been found to bind to water to prevent the availability of free water that bacteria need to grow and multiply.

Water activity may be defined as the ratio of the vapor pressure of water in a substance to the vapor pressure of pure water at the same temperature. Microorganisms require a minimum level of water activity in order to grow and, thus, water activity reduction can be used to control microbial growth.

Accordingly, it is desirable for the supplement to have a low water activity level. A humectant may be used to reduce the water activity level of the supplement. The humectant may bind to the free water molecules in the supplement and thereby reduce the water activity level. As non-limiting examples, the humectant may be nitric acid, dextrose, fructose, glycerol, glycine, glucose, honey, malic acid, salt, sorbitol, sucrose, and tartaric acid. A skilled artisan may select any suitable humectant, as desired. For example, the supplement can have a water activity level of 0.86.

Additionally, as may be presented in the claims herein, the language “consisting of” is intended to exclude any ingredient not specified in the claim. Accordingly, the supplement consisting of certain ingredients described hereinabove includes only those ingredients.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A supplement configured to be admixed in a product, the supplement comprising:
at least four ingredients from at least two groups with at least two ingredients selected from the first group including:
the first group containing anethole, benzaldehyde, carvacrol, caryophyllene, cineole, cinnamaldehyde, citral, citronellal, curcumin, farnesol, eugenol, limonene, linalool, methylglyoxal, pinene, terpineols, thujone, thymol, vanillin and zingiberene at a concentration between 50 and 50,000 parts per million in the product;
the second group containing betulinic acid, caprylic acid, chebulic acid, ferulic acid, flavonols, gingerol, hesperidin, isothiocyanates, mangostin, luteolin, myristic acid, oleuropein, pectolinarin, piperine, protocatechuic acid, pterostilbene, punicalagin, rhoifolin, sanguiin, and withanolides at a concentration between 100 and 100,000 parts per million in the product;
the third group containing acetic acid, citric acid, dextrose, fructose, glycerol, glycine, glucose, lactic acid, lactose, malic acid, propylene glycol, salt, sorbitol, sucrose, and tartaric acid at a concentration sufficient to change a water activity level or pH of the product.

2. The supplement of claim 1, wherein the supplement has a water activity level is less than 0.86.

3. The supplement in claim 1, wherein the supplement is admixed in a product.

4. The supplement of claim 3, wherein the supplement is added to a shake, thickened drink, weight loss drink, weight gain drink, milk, juice, carbonated water, water, honey, molasses, syrup, sauce, dressing, pudding, confection, or food.

5. The supplement in claim 1, wherein the supplement is configured to treat an infection from coronavirus.

6. The supplement in claim 1, wherein the supplement is configured to treat an infection from infectious bronchitis virus.

7. An antimicrobial supplement, preservative, honey product, natural gelling agent, or sweetener comprising at least one of honey, artificial honey, molasses, syrup, sorbitol, and glycerol;
admixed with at least three antivirals from the following list: an acid, an aldehyde, a catechin, curcumin, a flavanoid, a flavonoid, and a monoglyceride.

8. The supplement of claim 7, wherein the supplement has a water activity level is less than 0.86.

9. The supplement in claim 7, wherein the supplement is admixed in the product.

10. The supplement of claim 9, wherein the supplement is added to a shake, thickened drink, weight loss drink, weight gain drink, milk, juice, carbonated water, water, honey, molasses, syrup, sauce, dressing, pudding, confection, or food.

11. The supplement in claim 7, wherein the supplement is configured to treat an infection from coronavirus.

12. The supplement in claim 7, wherein the supplement is configured to treat an infection from infectious bronchitis virus.

13. An antimicrobial supplement, preservative, honey product, natural gelling agent, or sweetener comprising at least one of honey, artificial honey, molasses, and syrup;
admixed with a monoglyceride and at least one of an acid, a catechin, a gelling agent, a humectant, and a preservative.

14. The product of claim 13, comprising honey, monolaurin at 1%, and sorbic acid at 0.2%.

15. The product in claim 13, wherein the supplement is configured to treat an infection from coronavirus.
